(19) United States
(12) Patent Application Publication
(10) Pub. No.: US 2006/0134794 A1
(43) Pub. Date: Jun. 22, 2006

(54) Method of detecting the presence and/or progression of cancer

(76) Inventor: Stephen Webster, West Lafayette, IN (US)

Correspondence Address: Pepper Hamilton LLP;One Mellon Center, 50th Floor, 500 Grant Street, Pittsburgh, PA, 15219, US

(21) Appl. No.: 11/273,258

(22) Filed: Nov. 14, 2005

Related U.S. Application Data
(60) Provisional application No. 60/626,924, filed on Nov. 12, 2004.

(51) Int. Cl. G01N 33/00

(52) U.S. Cl. 436/64

(57) ABSTRACT

Silver (II) Oxide preferentially reacts with tNOX a cancer-specific ECTO-NOX protein of the surface of cancer cells. The combination of the cancer specificity of tNOX and the interaction of silver II oxide specified herein with tNOX offer new and novel therapeutic and diagnostic opportunities including but not restricted to coupling with one or more commercially available silver enhancement protocols for cancer detection as well as detection of an age-related ECTO-NOX form, designated ar-NOX.

### CROSS-REFERENCE TO RELATED APPLICATION

This application claims the benefit of U.S. Provisional Application No. 60/626,924 entitled “Silver (II) Oxide Targets a Cancer-Specific Protein, TNOX” filed Nov. 12, 2004, hereby incorporated by reference in its entirety.

There is a unique, growth-related family of cell surface hydroquinone or NADH oxidases with protein disulfide interchange activity referred to as ECTO-NOX protein (for cell surface NADH oxidases). One member of the ECTO-NOX family, designated tNOX (for tumor associated) is specific to the surfaces of cancer cells and the sera of cancer patients. The presence of the tNOX protein has been demonstrated for several human tumor tissues (mammary carcinoma, prostate cancer, neuroblastoma, colon carcinoma and melanoma), and serum analyses suggest a much broader association with human cancer.

NOX proteins are ectoproteins anchored in the outer leaflet of the plasma membrane. As is characteristic of other examples of ectoproteins (sialyl and galactosyl transferase, dipeptidylamino peptidase IV, etc.), the NOX proteins are shed. They appear in soluble form in conditioned media of cultured cells and in patient sera. The serum form of tNOX from cancer patients exhibits the same degree of drug responsiveness as does the membrane associated form. Drug-responsive tNOX activities are seen in sera of a variety of human cancer patients, including patients with leukemia, lymphomas or solid tumors (prostate, breast, colon, lung, pancreas, ovarian, liver). An extreme stability and protease resistance of the tNOX protein may help explain its ability to accumulate in sera of cancer patient to readily detectable levels. In contrast, no drug-responsive NOX activities have been found in the sera of healthy volunteers.

In addition, we have described an aging-related isoform (arNOX) of the ECTO-NOX family of proteins associated with sera and lymphocytes of patients about the age of 50 years or older capable of directly reducing ferric cytochrome c through the generation of superoxide. Previous findings demonstrate that the hyperactivity of the plasma membrane electron transport system results in an NADH oxidase activity capable of cell surface generation of reactive oxygen species. This would serve to propagate the aging cascade both to adjacent cells and to oxidize circulating lipoproteins. A unique feature of the aging-related ECTO-NOX isoform is that the generation of superoxide by this protein associated with aging is its inhibition by coenzyme Q. The findings provide a rational basis for the anti-aging activity of circulating coenzyme Q in the prevention of atherosclerosis and other oxidative changes in cell membranes and circulating lipoproteins.

Key identifying characteristics of the ECTO-NOX proteins as a family of functionally-related proteins are protease resistance (resistance to proteinase K digestion, for example), resistance to heating to temperatures between 70 and 80° C. and an oscillating activity with a temperature compensated period length of 24 minutes. The hydroquinone (NADH) oxidase activity and a protein disulfide-thiol interchange activity alternate to generate the 22 minutes period characteristic of tNOX. This characteristic alternation of activities is given as well by the Coenzyme-Q-inhibited aging isoform both at the lymphocyte cell surface and for a soluble serum form although the period length is 26 minutes rather than 22 minutes. Both the cancer-related and the age-related NOX activities have been purified and the activities of the purified proteins also oscillate.

### BRIEF SUMMARY OF THE INVENTION

One embodiment of the present invention provides a silver (II) oxide (AgO)-based serum color test specific for cancer-related (tNOX) ECTO-NOX proteins. The test may comprise silver (II) oxide and serum. The test may further comprise (−)-epigallocatechin-3-gallate, nickel chloride and/or silver enhancer.

A further embodiment of the present invention provides a method of detecting the presence of cancer-related ECTO-NOX proteins, including tNOX. The method may include the mixing solid silver (II) oxide with a serum sample from a patient. The sample may then be incubated and then silver enhancer may be added. The method may further include the steps of adding (EGCg) and adding nickel chloride prior to incubation. Following addition of the silver enhancer, the color may be measured following development.

Another embodiment of the present invention provides a silver (II) oxide-based serum color test specific for aging-related (arNOX) ECTO-NOX proteins to monitor cancer presence and progression. The test may comprise silver (II) oxide and serum. The test may further comprise (−)-epigallocatechin-3-gallate, nickel chloride and/or silver enhancer.

A further embodiment of the present invention provides a method of detecting the presence of age-related ECTO-NOX proteins, including arNOX. The method may include the mixing solid silver (II) oxide with a serum sample from a patient. The sample may be incubated and silver enhancer may be added. The method may further include adding (EGCg) and adding nickel chloride prior to incubation. Following addition of the silver enhancer, the color may be measured following development.

### BRIEF DESCRIPTION OF THE DRAWINGS

For a fuller understanding of the nature and advantages of the present invention, reference should be had to the following detailed description taken in connection with the accompanying drawings, in which:

FIG. 1 shows inhibition of a recombinant form of tNOX (nus tNOX with a nus tag to reduce aggregation and as an aid to purification) by 100 μM silver (II) oxide (AgO) in 1.7% phosphoric acid added after 45 minutes (panel B) and the assay was continued for and additional 45 minutes. In panel A, an equivalent amount of phosphoric acid was added. The assay measured oxidation of NADH (decrease in A₃₄₀) in a standard NOX assay at 25° C. The characteristic 5-maxima pattern of NOX activity was seen with a repeat every 22 minutes (double arrows). In the presence of the AgO, the activity was inhibited completely.

FIG. 2 is similar to FIG. 1 except that a preparation of solubilized ECTO-NOX activities released from the surface of HeLa cells by treatment at pH 5 (del Castillo-Olivares et al., Arch. Biochem. Biophys. 358: 125-140, 1998) was used as the enzyme source. The AgO was dissolved in DMSO added at a final concentration of AgO of 1 mM. The final concentration of DMSO added in both A and B after 45 minutes was 0.1%. The assays were then continued for and additional 45 minutes. The HeLa extracts contain both the constitutive CNOX (single arrows denoting a 24 minutes period length) and two activity maxima separated by 6 minutes and labeled \textcircled{1} and \textcircled{2} which are tNOX (double arrows). With addition of AgO, both tNOX activities are inhibited while the constitutive CNOX remain unaffected. Values are based on three experiments and report means±standard deviations to establish statistical reliability of the data.

FIG. 3 represents the response of total NOX (NADH oxidase) activity of the preparation of FIG. 2 as a function of the logarithm of AgO concentration. The AgO was dissolved in 1.7% phosphoric acid and the dilutions were prepared in water. The diluted phosphoric acid in the absence of AgO was without effect on the NOX activity (not shown). The preparations contained approximately equal amounts of constitutive CNOX (not inhibited) and tNOX (inhibited) suggesting that the tNOX was completely inhibited at an AgO concentration of 100 μM (10⁻⁴M). The EC₅₀ of tNOX inhibition was estimated to be about 5 μM.

FIG. 4 is similar to FIG. 2 except that NOX activity was assayed with intact HeLa cells (10⁶). The AgO was dissolved in 1.7% phosphoric acid and added after 45 minutes (panel B). The cells of panel A received only the phosphoric acid at a final concentration of 0.0017%.

FIG. 5 presents results from an assay using 2×10⁶ human mammary adenocarcinoma (BT-20) cells carried out as described for FIG. 2. Panel A (no addition) received only the phosphoric acid (0.0017%). The 100 μM AgO inhibited the two activity maxima associated with tNOX (double arrows) labeled \textcircled{1} and \textcircled{2} and was without effect on the constitutive CNOX (single arrows).

FIG. 6 is as for FIG. 5 except that the assay was with 10⁶ human mammary epithelial (non-cancer) MCF-10A cells. These cells lacked tNOX as their surface and contained predominantly a constitutive CNOX (single arrows) which was not inhibited by the 100 μM AgO added in 1.7% phosphoric acid. The MCF-10A cells were late passage cells and contained, as well, an age-related NOX (arNOX) with a period length of 26 minutes. This activity was inhibited by the AgO as shown in Panel B.

FIG. 7 is similar to FIG. 6 except that plasma membranes isolated from etiolated hypocotyls (stems) of soybean (Glycine max) were the enzyme source. The plasma membranes were solubilized in 0.1% Triton X-100 overnight prior to assay. The soybean plasma membranes contain only a constitutive CNOX which is unresponsive to AgO.

FIG. 8 shows proportionality to amount of sera between 5 and 30 μl.

FIG. 9 is the reflectance reading as a function of amount of sera between 30 and 70 μl. Pooled cancer is compared to an 81 year old non-cancer female. Above 30 μl, other reagents become limiting and no further increase in reflectance readings were observed.

FIG. 10 is the reflectance reading as a function of amount of sera between 30 and 70 μl. Pooled cancer is compared to a non-cancer 73 year old male and an 89 year old female.

FIG. 11 displays results of assays where the sera amount remained constant at 30 μl but the ratios of pooled cancer to non-cancer (73 M) sera were varied within the 30 μl. Beginning at 10 μl, the reflectance readings were proportional to the amount of cancer sera present in the mixture.

FIG. 12 displays results of assays where the sera amount remained constant at 30 μl but the ratios of pooled cancer to non-cancer (65 F) sera were varied within the 30 μl. Beginning at 10 μl, the reflectance readings were proportional to the amount of cancer sera present in the mixture.

FIG. 13 documents the reflectance readings as a function of the amount of solid AgO added to the reaction. The optimum amount was verified as 5 mg. Here the values for the pooled cancer were positive and values averaged for the three non-cancer samples were negative. The points near the origin were for a 1:100 dilution of the AgO.

FIG. 14 shows results of adding (−)-epigallocatechin-3-gallate (EGCg) to the silver reagent.

FIG. 15 shows results of addition of (−)-epigallocatechin-3-gallate (EGCg) to the silver reagent.

FIG. 16 is a composite figure summarizing results from a large number of experiments with pooled cancer sera to optimize the amount of EGCg added to the reaction.

FIG. 17 shows results to verify the amount of nickel chloride added to the assay as 30 μl of 0.25 M.

FIG. 18 shows the effect of incubation time before addition of silver enhancer comparing pooled cancer and pooled non-cancer sera.

FIG. 19 compares 4 different sera from individual non-cancer individuals with results similar to those with pooled non-cancer sera of FIG. 10.

FIG. 20 shows time of incubation prior to addition of the silver enhancer reagent between 15 and 30 minutes.

FIG. 21 gives reflectance readings as a function of the amount of silver enhancer for the pooled cancer sample.

FIG. 22 shows response to the amount of silver enhancer used in the assay.

FIG. 23 shows response to amount of silver enhancer for pooled cancer and sera from a prostate and an ovarian cancer patient compared to non-cancer patients.

FIG. 24 compares breast and liver cancer and leukemia with 3 non-cancer sera (Panel A) and sera from a brain cancer patient with sera from a 82 year old non-cancer male (Panel B).

FIG. 25 provides information of the reflectance readings as a function of the time of development in seconds after addition of the silver reducer and before adding the stop reagent for pooled cancer, 5 individual cancer sera and sera from a 51 year old non-cancer male.

FIG. 26 shows an apparent lack of correlation between the amount of tNOX dimer (Panel A) or tNOX 3 monomer and the reflectance readings for a series of cancer patients.

FIG. 27 gives the correlation between total tNOX amount (μg/100 μl serum) and the A/(L+B) values.

FIG. 28 is a standard NOX assay using recombinant tNOX with a GST (glutathione-S-transferase) tag to facilitate purification. Upon addition of 50 μl (5×10⁴) beads with AgO attached after 48 min, the beads were removed by centrifugation and the assay of enzymatic activity was resumed. The tNOX activity was not measurable and the tNOX protein is presumed to have been bound to the beads.

FIG. 29 is as in FIG. 28 except that the comparison with magnetic beads to which no AgO was bound (A) shows that such beads did not reduce tNOX activity of the preparations to demonstrate that, without the AgO, the magnetic beads did not bind tNOX.

FIG. 30 (A) beads without the AgO (blank beads) were present from the beginning of the assay, with no effect on tNOX activity while in (B), beads with AgO bound were present from the beginning of the assay and tNOX activity was reduced to base-line levels.

FIG. 31 illustrates HeLa S (human cervical carcinoma cells) cells treated with either blank beads (magnetic beads without AgO) or AgO beads (magnetic beads with AgO) to show specific binding of the magnetic beads to tNOX bound to the surface of the HeLa cells. The HeLa cells with beads bound could then be collected by exposure to a magnetic field (e.g. a hand-held magnet or a device designed to collect cells with bound magnetic beads).

FIG. 32 gives the correlation between serum A/(L+B) values for the color test and arNOX based on measurement of generation of superoxide (superoxide dismutate-inhibited reduction of ferrocytochrome c). Open symbols are for cancer patients. Solid symbols are for volunteers not diagnosed as having cancer. For non-cancer patients, an almost linear correlation was observed. The presence of ar-NOX in serum (as for a cancer test) can be reduced by soon after centrifugation of the serum following blood collection.

FIG. 33 shows the distribution of serum A/(L+B) values for volunteers not diagnosed as having cancer according to age. Values are greatest in the 61 to 70 year-old age group which have the highest age-related ECTO-NOX (arNOX) activities and the greatest risk of atherogenic disease.

FIG. 34 reports color development time (seconds) comparing pooled sera from non-cancer, non-aged volunteers (open symbols, dotted lines), pooled sera from non-cancer, aged volunteers expressing high levels of arNOX (open triangles, dotted lines) and pooled sera sample from cancer patients (solid symbol and solid lines) showing more rapid reaction of arNOX with AgO in the color test than with tNOX.

FIG. 35 provides a relationship between hemoglobin content of serum due to hemolysis (A₄₁₀-A₄₄₀) and A/(L+B) color test values. Values were generated by addition of red blood cell lysate in small amounts to normal patient sera prior to testing. A₄₁₀-A₄₄₀ values greater than about 0.15 result in false positive A/(L+B) values.

### DETAILED DESCRIPTION OF INVENTION

Before the present compositions and methods are described, it is to be understood that this invention is not limited to the particular processes, compositions, or methodologies described, as these may vary. It is also to be understood that the terminology used in the description is for the purpose of describing the particular versions or embodiments only, and is not intended to limit the scope of the present invention which will be limited only by the appended claims.

It must also be noted that as used herein and in the appended claims, the singular forms “a”, “an”, and “the” include plural reference unless the context clearly dictates otherwise. Thus, for example, reference to a “cell” is a reference to one or more cell and equivalents thereof known to those skilled in the art, and so forth. Unless defined otherwise, all technical and scientific terms used herein have the same meanings as commonly understood by one of ordinary skill in the art. Although any methods and materials similar or equivalent to those described herein can be used in the practice or testing of embodiments of the present invention, the preferred methods, devices, and materials are now described. All publications mentioned herein are incorporated by reference in their entirety. Nothing herein is to be construed as an admission that the invention is not entitled to antedate such disclosure by virtue of prior invention.

As used herein, the term “about” means plus or minus 10% of the numerical value of the number with which it is being used. Therefore, about 50% means in the range of 45%-55%.

One embodiment of the present invention provides a silver (II) oxide (AgO)-based serum color test specific for cancer-related (tNOX) ECTO-NOX proteins. The test may comprise silver (II) oxide and serum from a patient. In one embodiment, the test utilizes between about 5 to about 70 μl of serum, more preferably about 30 μl of serum, and preferably about 0.5 to about 50 mg of AgO, more preferably about 5 mg of AgO. The test may further comprise (−)-epigallocatechin-3-gallate (EGCg), preferably from about 0.25 mM to about 10 mM, more preferably about 0.25 to about 1 mM. The test may further comprise nickel chloride, preferably about 0.25 to about 3 M, more preferably about 0.25 M. The test may further comprise a silver enhancer, preferably about 5 to about 70 μl, more preferably from about 30 to about 45 μl. The test may further comprise a stop reagent, preferably 80 mM sodium thiosulfate pentahydrate.

Another embodiment of the present invention provides a method of detecting the presence of cancer-related ECTO-NOX proteins, including tNOX. The method may include the mixing solid silver (II) oxide with a serum sample from a patient. The sample may then be incubated and then silver enhancer may be added. The method may further include the steps of adding (EGCg) and adding nickel chloride prior to incubation. Following addition of the silver enhancer, the color may be measured following development. In one embodiment, the method includes mixing soluble AgO with EGCg, preferably from about 0.25 to about 1 mM. The method further comprises the step of mixing about 30 μl of serum from a patient and about 30 μl of 250 mM nickel chloride. The method further comprises incubating the mixture at room temperature for about 15 minutes. Optionally, the method may further comprise the step of adding a silver enhancer, preferably about 30 μl of silver enhancer. The method further comprises a step of development, wherein color is allowed to develop for about 25 seconds. Optionally, the method may further comprise the step of adding a stop reagent, preferably 30 μl of 80 mM sodium thiosulfate pentahydrate, to the mixture. The method further comprises the step of observing the color to determine the presence of tNOX, which is indicative of the presence of cancer.

Another embodiment of the present invention provides a silver (II) oxide-based serum color test specific for aging-related (arNOX) ECTO-NOX proteins to monitor cancer presence and progression. The test may comprise silver (II) oxide and serum. The test may further comprise (−)-epigallocatechin-3-gallate, nickel chloride and/or silver enhancer. In another embodiment, the method may comprise the additional step of centrifugation of the serum prior to adding to the AgO mixture.

A further embodiment of the present invention provides a method of detecting the presence of age-related ECTO-NOX proteins, including arNOX. The method may include the mixing solid silver (II) oxide with a serum sample from a patient. The sample may then be incubated and then silver enhancer may be added. The method may further include the steps of adding (EGCg) and adding nickel chloride prior to incubation. Following addition of the silver enhancer, the color may be measured following development.

In a preferred embodiment, the method utilizes only about 30 μl of serum (0.03 ml)=about ½ drop) and can be completed in less than 20 minutes. Reagent shelf life is about 2 to about 4 weeks. In certain embodiments, an orange to dark red color indicates cancer and non-cancer specimens remain clear or light yellow. The color may be stabilized by the addition of a stop reagent. We have used a reflectance spectrophotometer to record LAB values which are measures of intensity and quality of the colors generated. A/(L+B) values for cancer specimens are positive (0.171±0.09) and for non-cancer specimens are negative (−0.025±0.009).

False positives indicating cancer may result from the presence of the age-related ECTO-NOX protein (arNOX) or from hemolytic release of hemoglobin. In order to reduce false positives, the sample may be centrifuged immediately after collection under conditions to minimize both arNOX and hemolysis.

In a further embodiment, a protocol was developed to bind AgO to magnetic beads to show binding of AgO to tNOX. Addition of the AgO-derivatized magnetic beads but not magnetic beads without AgO, remove tNOX (as well as arNOX) from serum based on assays of activity and gel electrophoresis. HeLa cells which express tNOX at their surface bound the AgO-derivatized beads but not beads without AgO to provide a method to separate cancer cells from blood by virtue of the bound beads. With prolonged (several hours) of standing of serum samples at room temperature, the color test provides a measure of arNOX which may have utility as a clinical measure of atherogenic risk.

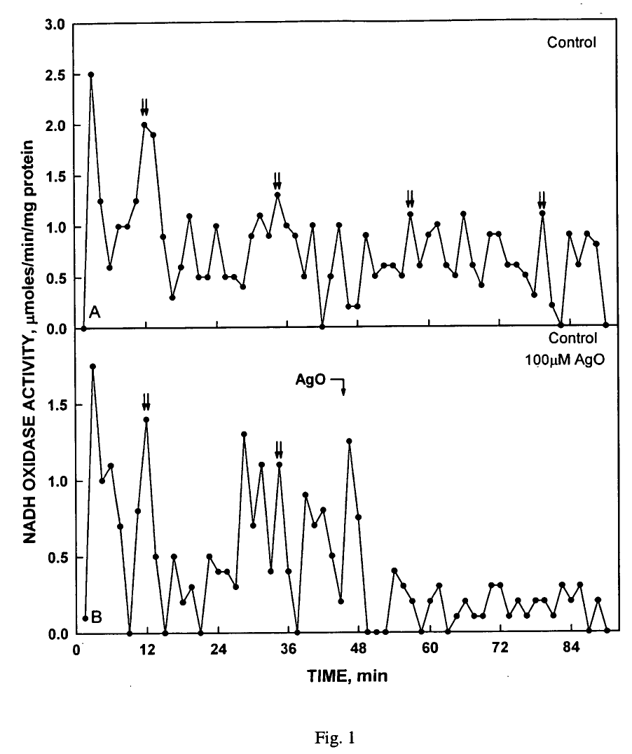

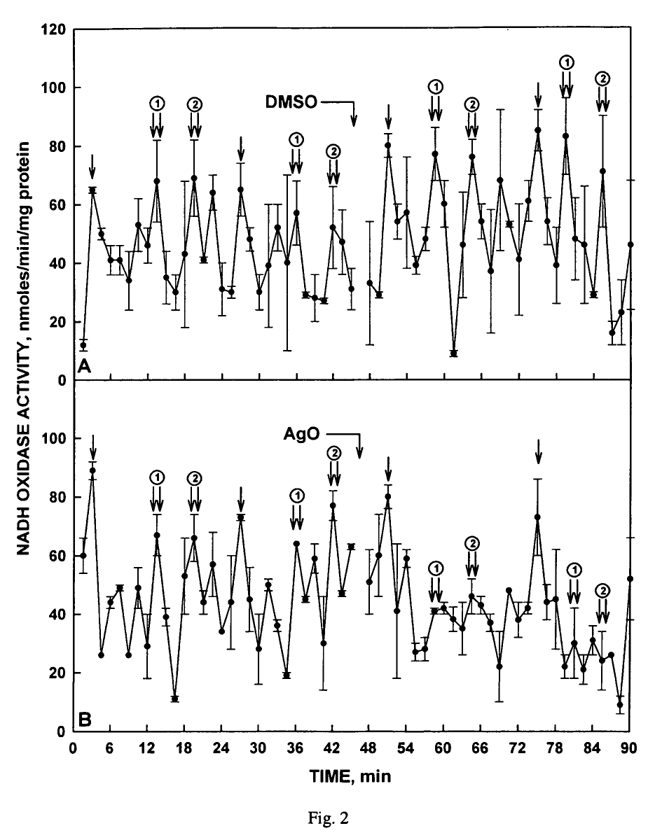

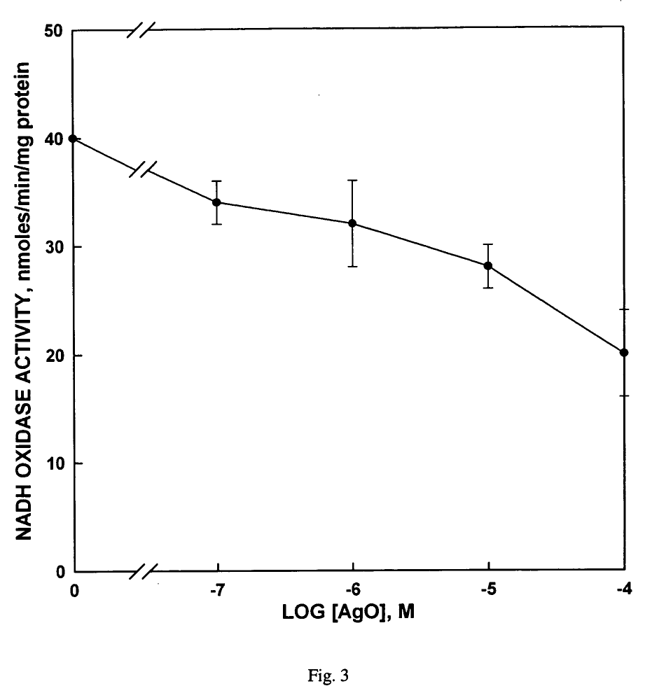

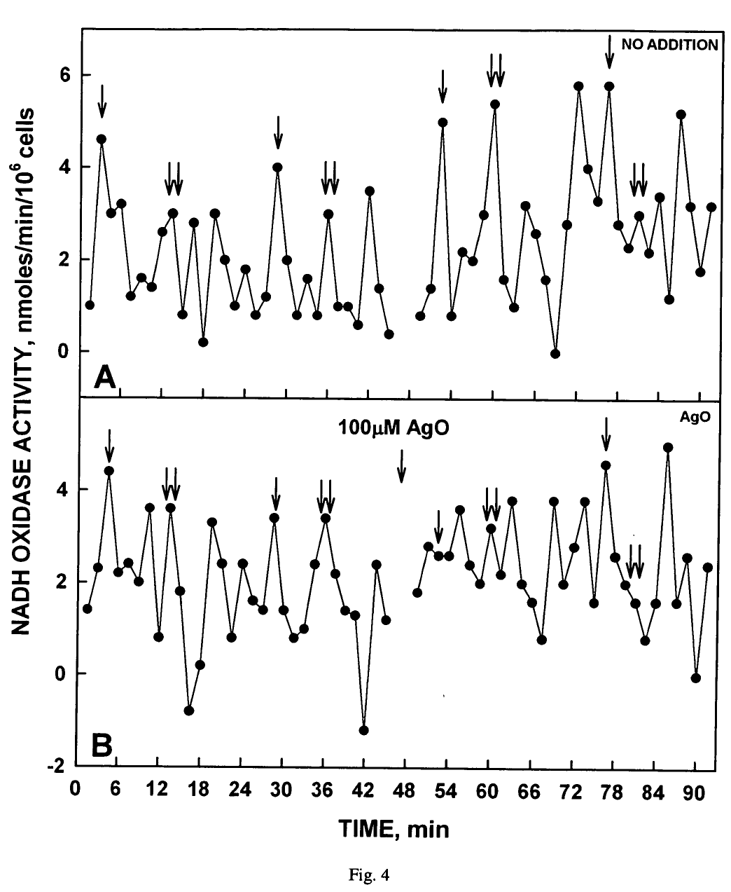

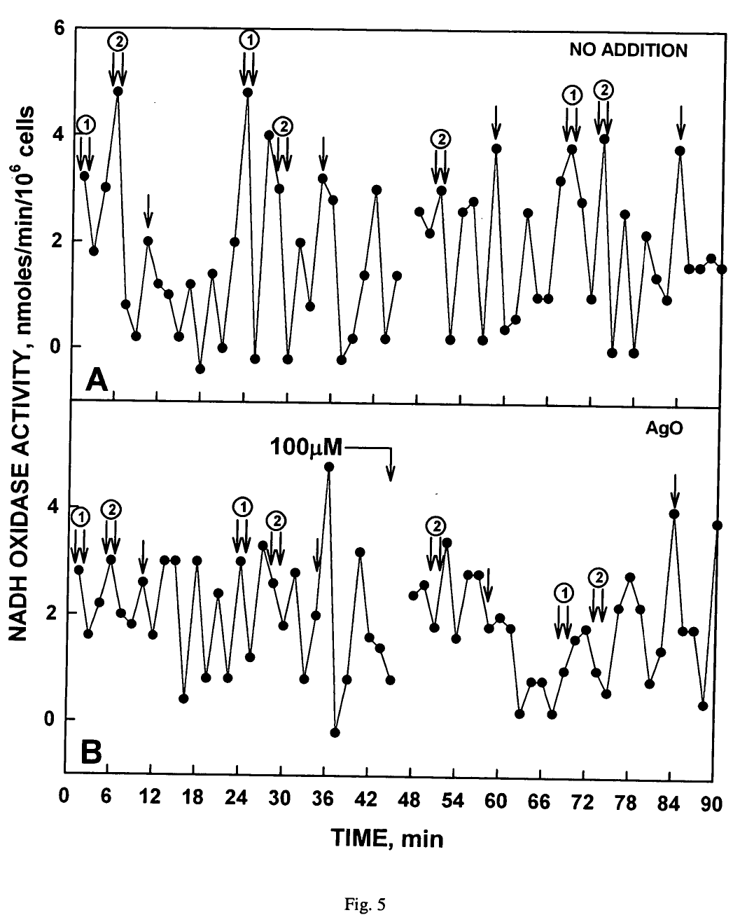

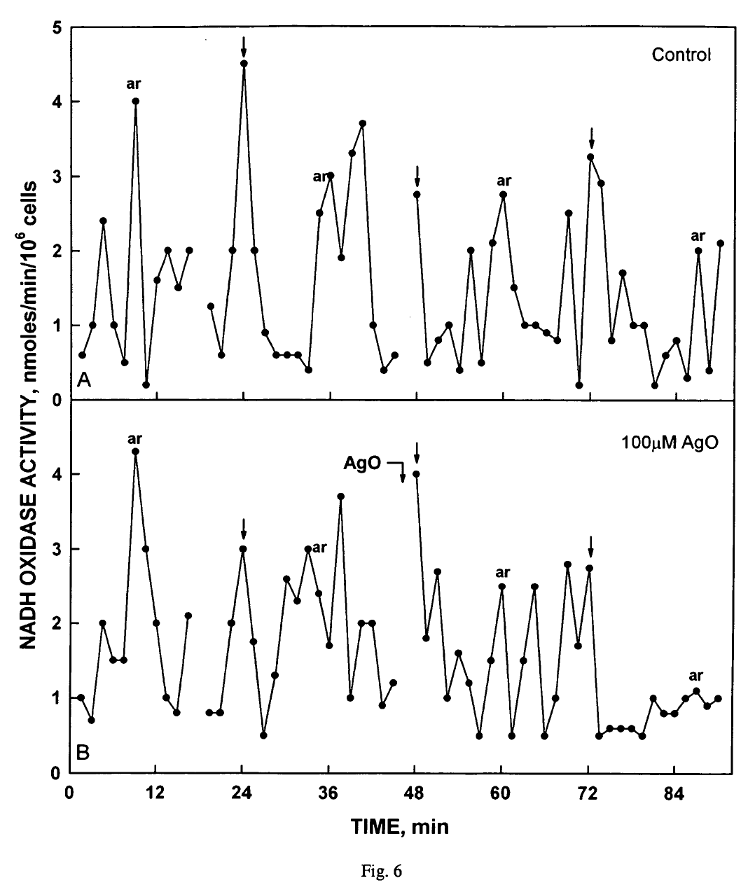

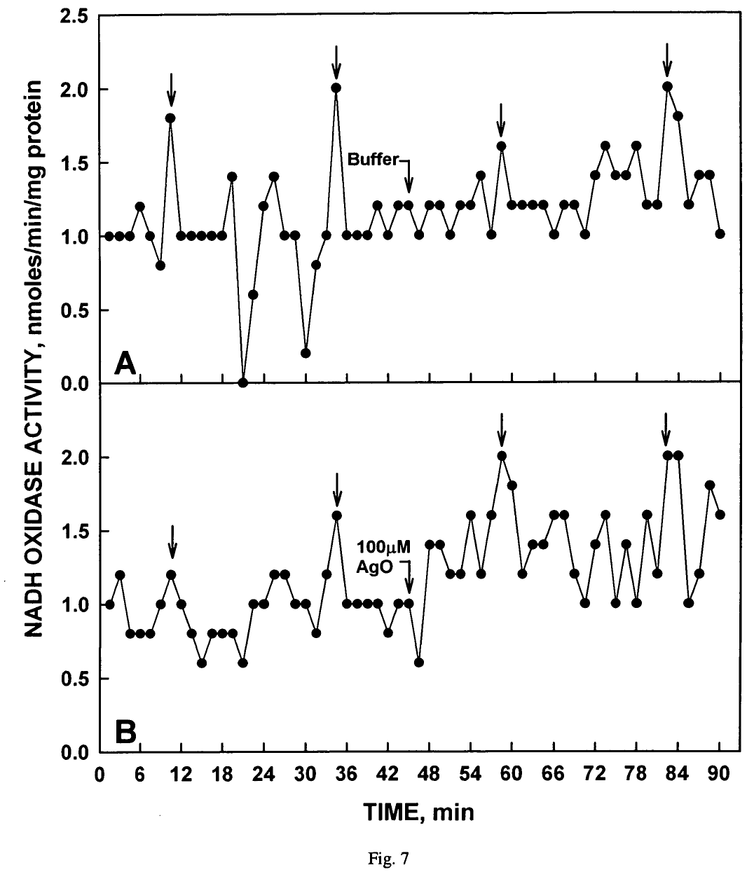

FIGS. 1 to 5 show the inhibition of the cancer ECTO-NOX isoforms tNOX by AgO (double arrows) with a 22 minutes period (FIG. 1 is recombinant tNOX assayed separately to unequivocally show inhibition) but not of the constitutive ECTO-NOX (CNOX) with a period length of 24 minutes for ECTO-NOX activities released from the surface of HeLa cells (FIGS. 2 and 3) and human cervical carcinoma (HeLa S) (FIG. 4) or human mammary carcinoma BT-20 cells (FIG. 5) grown in culture. ECTO-NOX activities of non-cancer human mammary MCF-10A cells which lack tNOX entirely at their cell surface were unaffected by AgO (FIG. 6) as were ECTO-NOX activities of soybean plasma membranes which also lack tNOX (FIG. 7).

One embodiment of this invention utilizes the ability of AgO to bind to tNOX of cancer cells and not to constitutive ECTO-NOX proteins of non-cancer cells as the basis for a strategy for the colorimetric estimation of cancer presence. In a preferred embodiment, the protocol describes a unique combination of soluble AgO with (−)-epigallocatechin-3-gallate (EGCg), serum, nickel chloride (NiCl₂) and silver enhancer to effect color development. Color may be measured using a reflectance spectrophotometer and expressed as the ratio of A/(L+B).

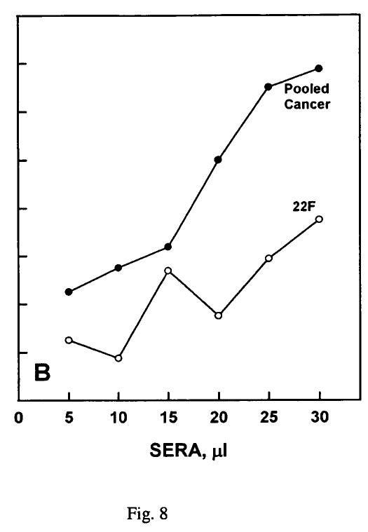

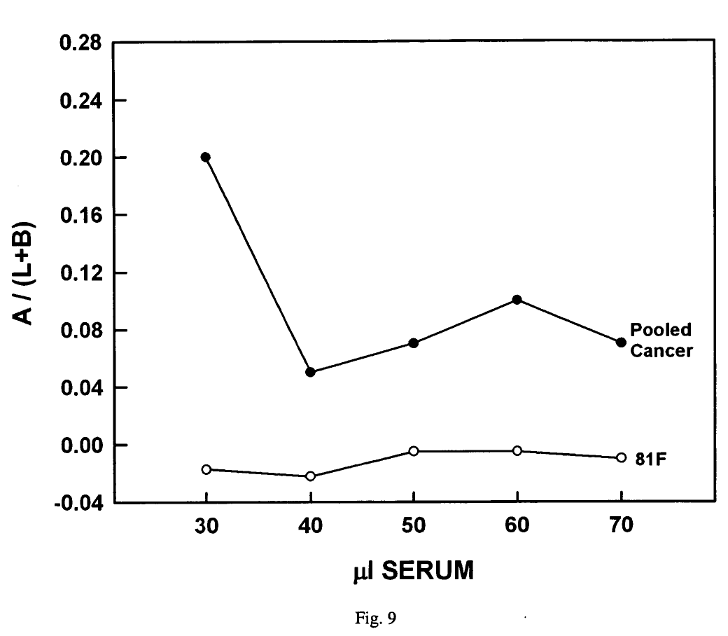

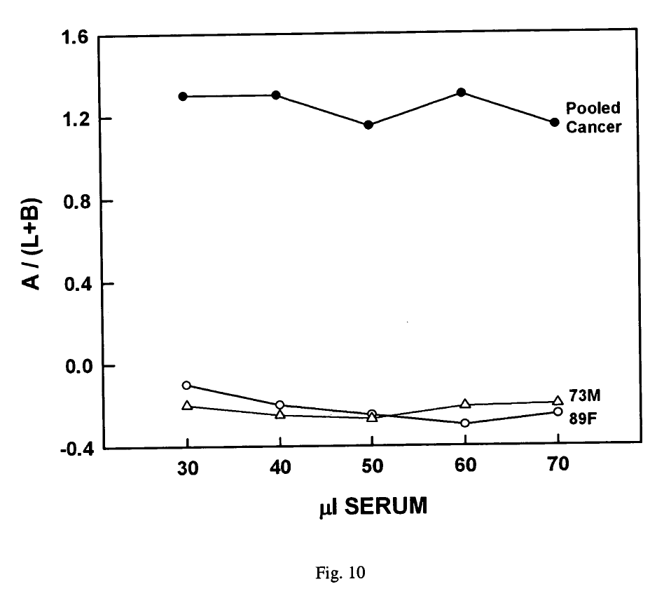

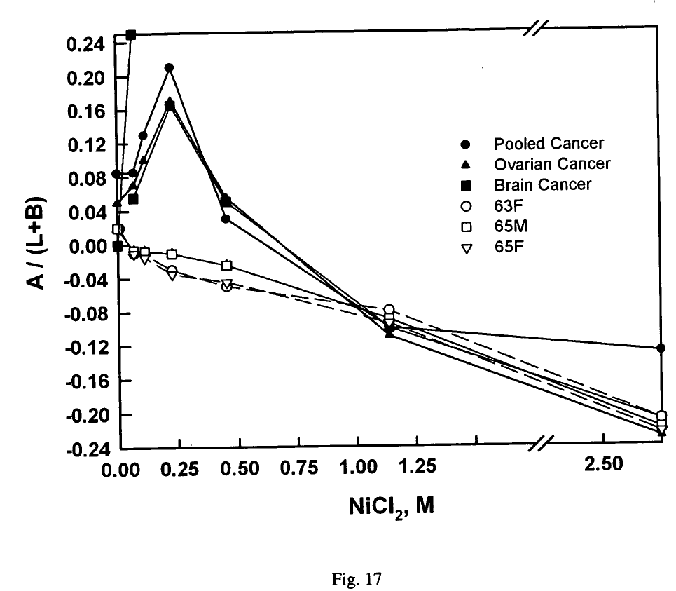

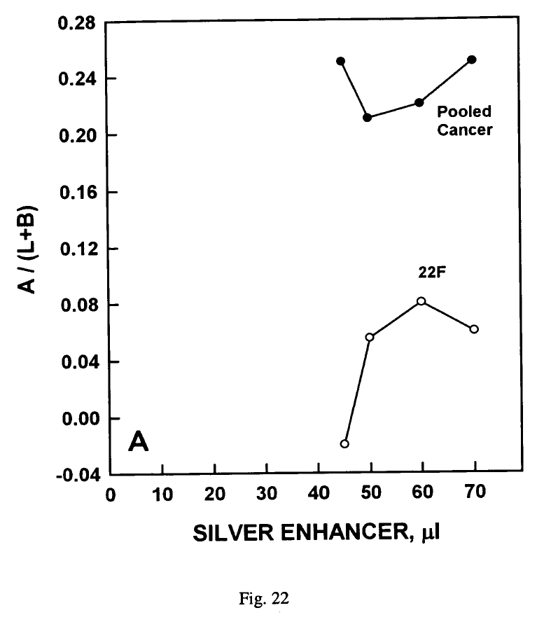

Each of the solutions and conditions of the protocol of Example 1 has been optimized with the results being illustrated in FIGS. 8 through 27. FIG. 8 shows linearity of color development up to and including 30 μl of cancer sera compared to sera of a 22 year old female non-cancer patient. Amounts of sera greater than 30 μl did not result in either increased color development nor in an increased differential between cancer and non-cancer (81 year old female) (FIG. 9). The utility of the 30 μl sample size is further validated for a pooled cancer sample compared to two non-cancer sera samples (73 year old male and 89 year old female). Beyond 30 μl, other components of the assay mixture become limiting and the color level plateaus (FIG. 10).

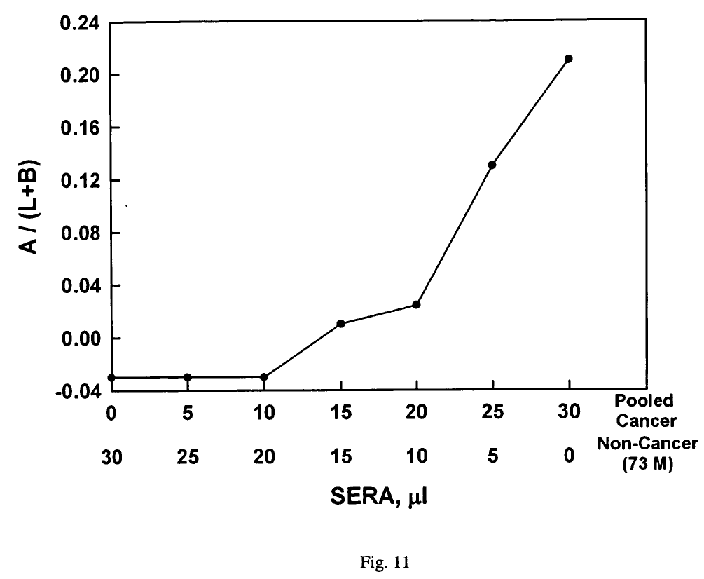

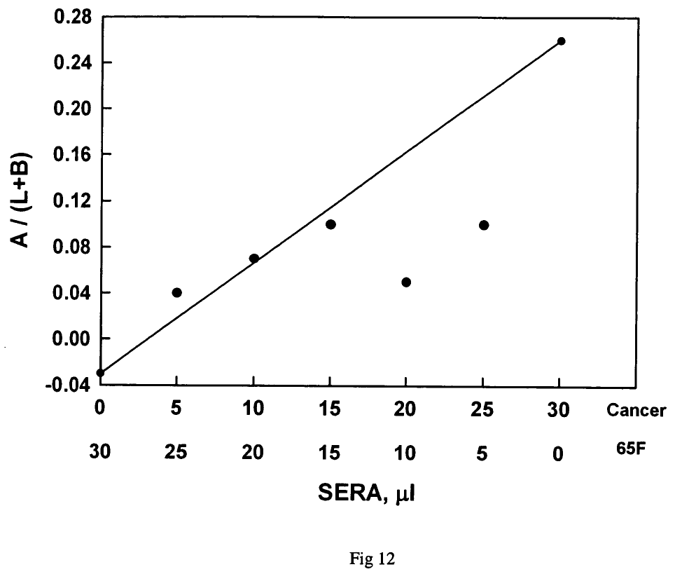

FIG. 11 displays results of assays where the sera amount remained constant at 30 μl but the ratios of pooled cancer to non-cancer (73 year old male) sera were varied within the 30 μl. Beginning at 10 μl, the reflectance readings were proportional to the amount of cancer sera present in the mixture. These findings demonstrate proportionality to tNOX of cancer sera and provide the basis for the tests ability to quantitated serum levels of tNOX. Similar results are shown in FIG. 12 where cancer sera and sera from a 65 year old female were mixed.

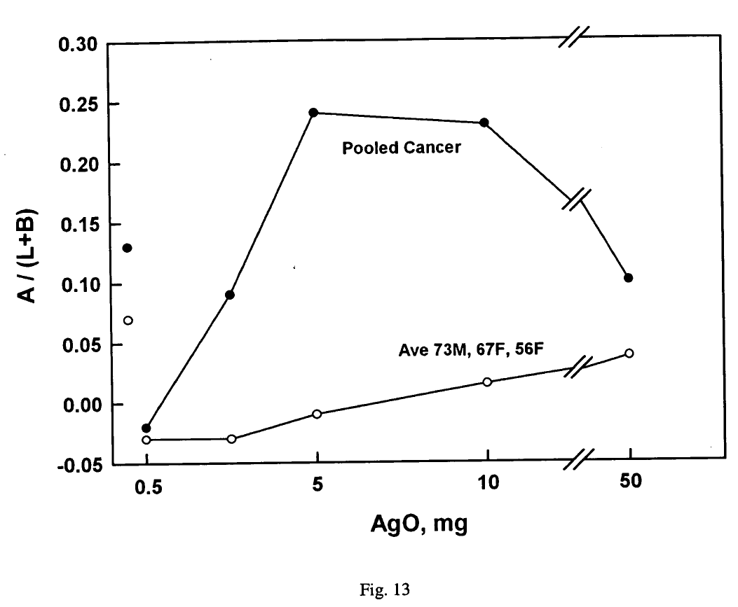

The assay used solid silver (II) oxide (AgO) as the source of reactive silver. The optimum amount for 30 μl of sera was demonstrated to be 5 mg (FIG. 13), the amount specified by the protocol of Example 1.

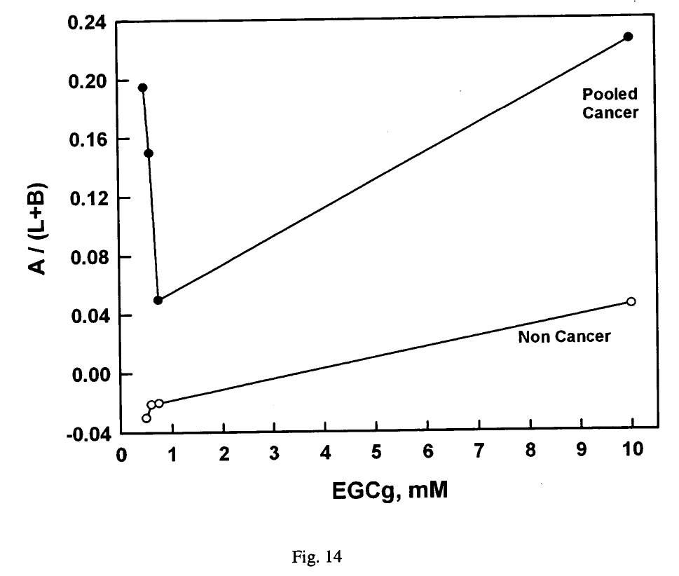

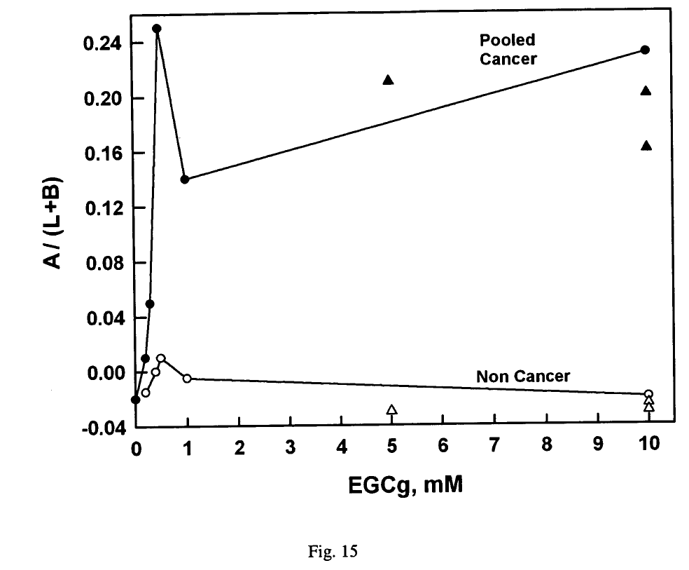

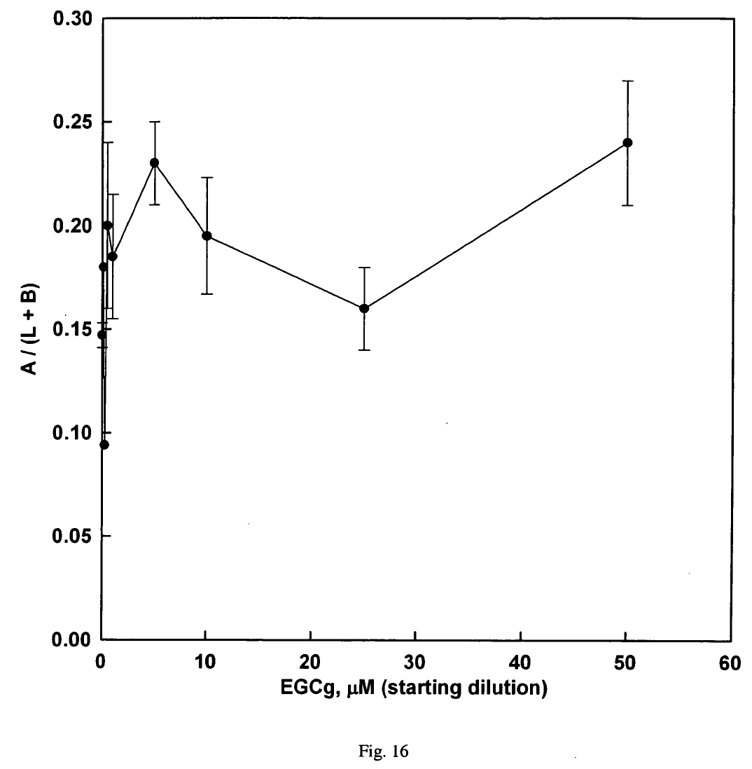

A further unique feature of the protocol of Example 1 is the addition of (−)-epigallocatechin-3-gallate (EGCg) to the silver oxide to increase sensitivity. The optimum was between 0.25 and 1 mM (FIG. 14). Similar measurements of FIG. 15 place the optimum as between 0.5 m as 0.5 mM or 10 mM. FIG. 16 is a composite figure summarizing results from a large number of experiments with pooled cancer to optimize the amount of EGCg added to the reaction. The optimum on average was between 0.25 and 1 μl consistent with the amounts specified in the protocol of Example 1.

Nickel chloride is not essential to the protocol (see Table 1), but 30 μl of 0.25 M nickel chloride enhances the color development (FIG. 16).

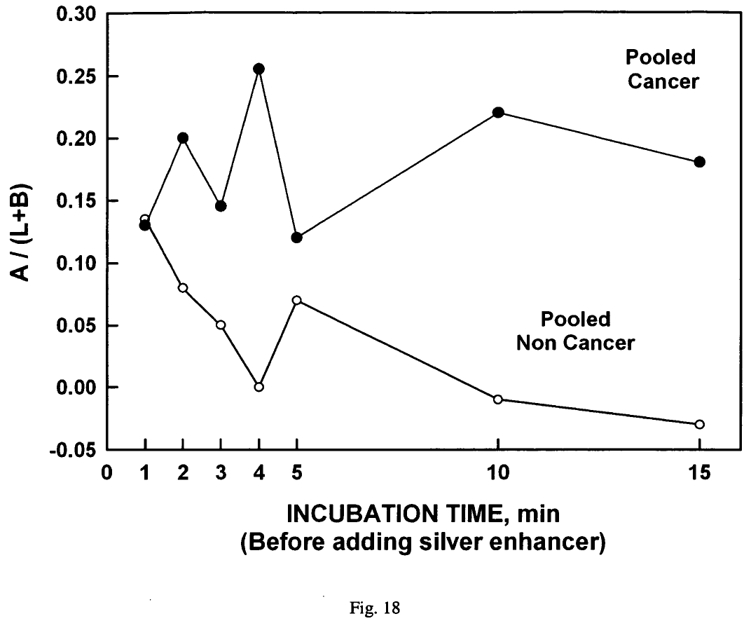

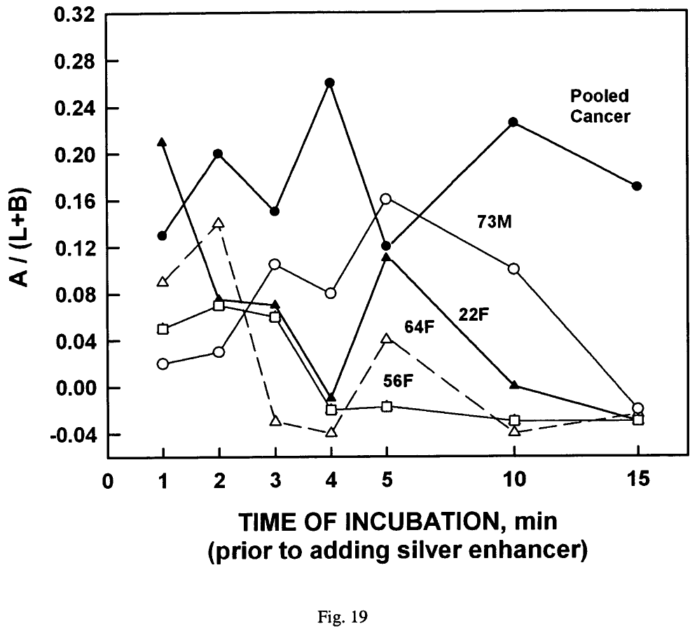

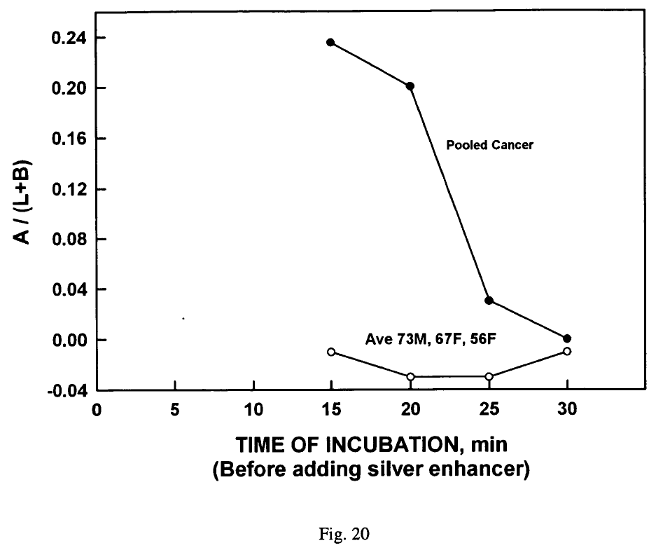

The protocol of Example 1 specifies that the sample be incubated prior to addition of silver enhancer. The incubation time was not critical. A time of 15 minutes was selected as when A/(L+B) values of non-cancer sera became negative and A/(L+B) values of pooled cancer sera remained positive (FIG. 18). This is illustrated more clearly in FIG. 19 where the sera from four non-cancer patients (73 year old male, 22 year old female, 64 year old female and 56 year old female) did not become negative until 15 minutes of incubation with the pooled cancer sera remaining positive throughout. Increasing the time beyond 15 minutes resulted in loss of color from the cancer sera (FIG. 20) such that the 15 minutes incubation time was adopted in the protocol of Example 1.

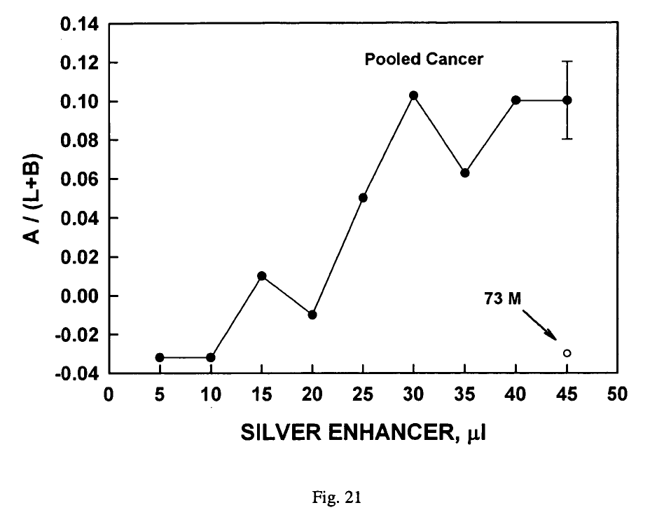

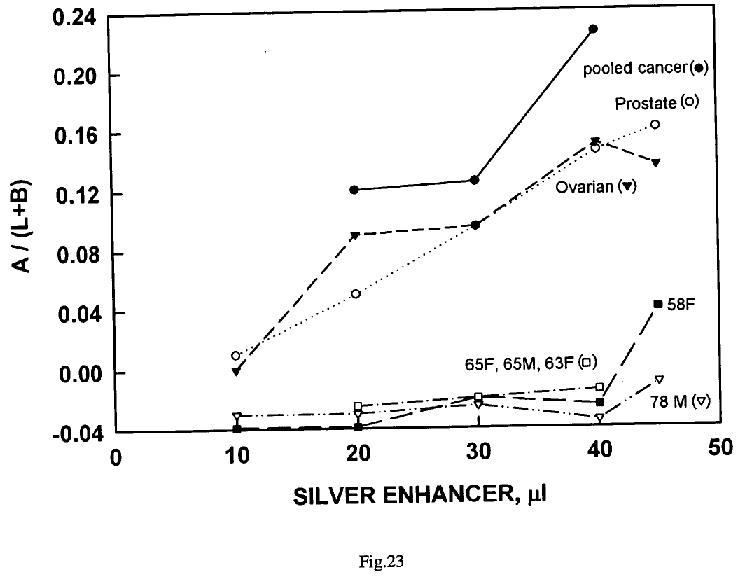

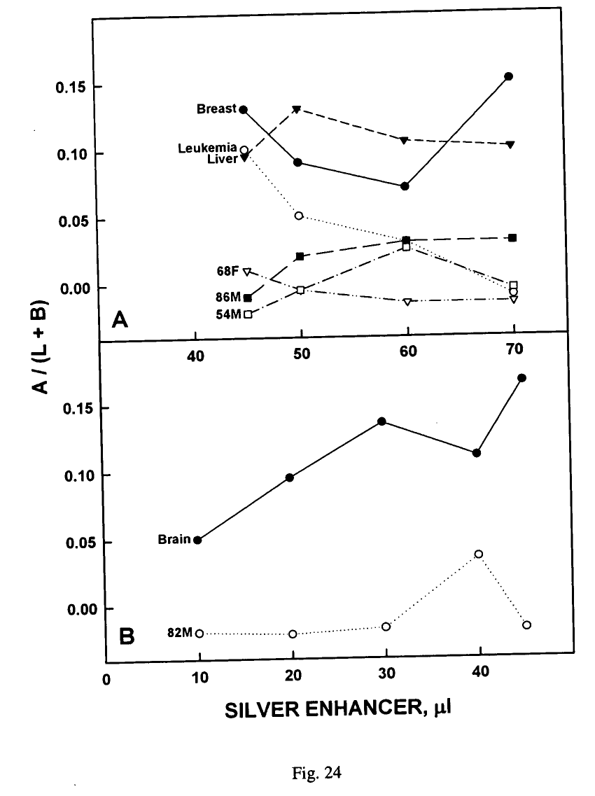

Silver enhancer increased for color development. The optimum amount was in the range of 30 to 45 μl (FIG. 21) with a development time of 25 to 30 seconds. Adding more silver enhancer did not appear to improve the color amount of the cancer sera and resulted in the color amount of the non-cancer sample becoming positive. The optimum amount of silver enhancer (between 30 and 40 μl) is shown in FIG. 23 for both pooled cancer sera and individual sera from a prostate and an ovarian patient compared to non-cancer controls. Results for breast cancer, leukemia, liver cancer and brain cancer are given in FIG. 24 compared to sera from four non-cancer individuals.

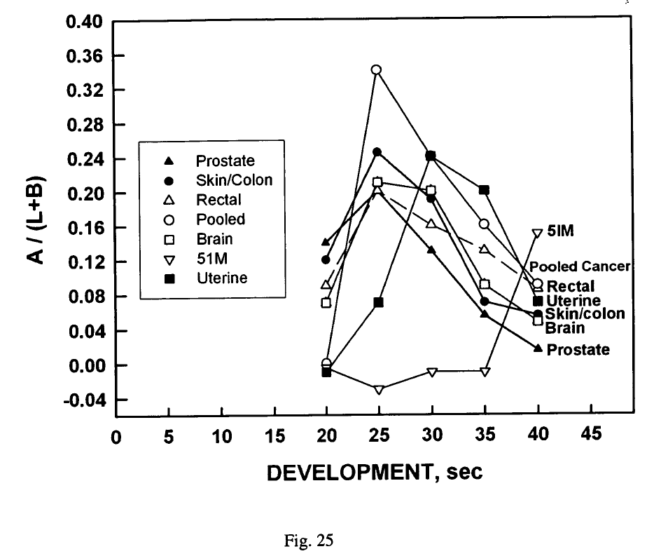

The development time after addition of silver enhancer was relatively critical (FIG. 25). For pooled cancer, and sera from rectal, uterine, skin, colon, brain and prostate patients, a 30 seconds development time was optimal to achieve a high cancer value and a negative non-cancer serum value compared to sera of a 51 year old male non-cancer individual.

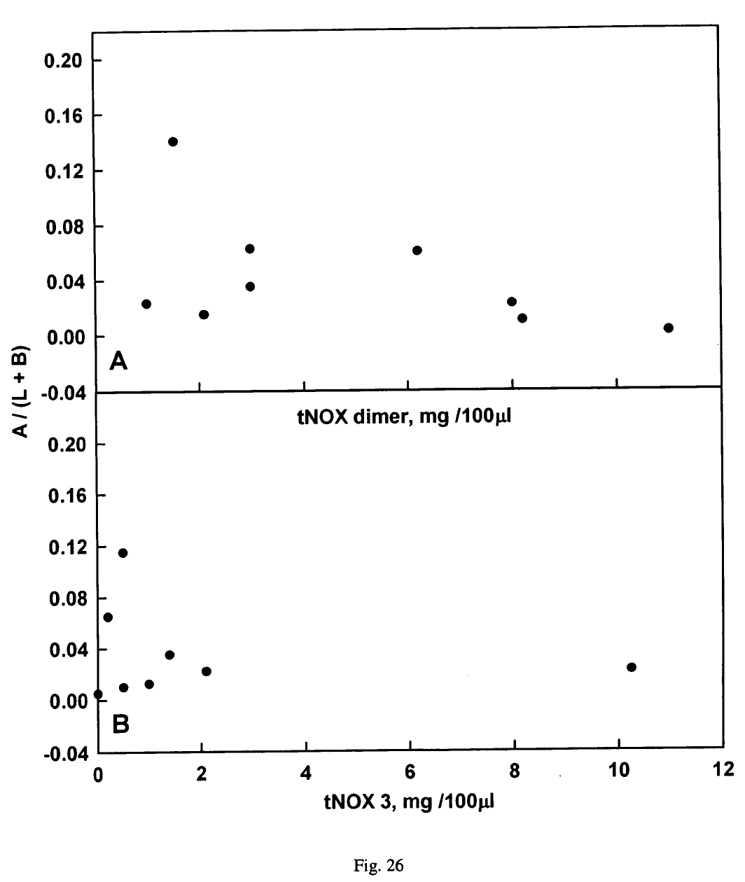

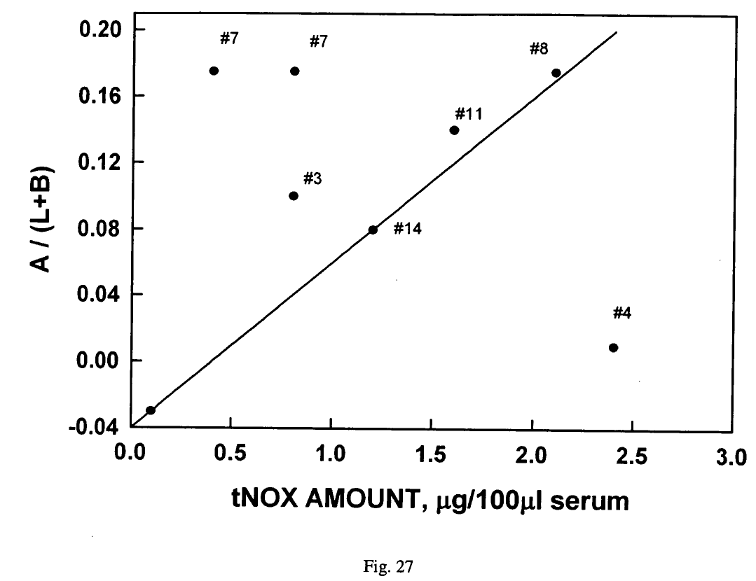

FIG. 26 shows an apparent lack of correlation between the amount of tNOX dimer or tNOX 3 monomer and the reflectance readings for a series of cancer patients. The different isoforms were quantitated after resolution on polyacrylamide gels and Western blotting with tNOX-specific recombinant antisera. A correlation between total tNOX amount (μg/100 μl serum) and the A/(L+B) values is given in FIG. 27 also determined by polyacrylamide gel electrophoresis and Western blotting. While not wishing to be bound by theory, the values for number 7 may be a result of the presence of ECTO-NOX isoforms other than tNOX 1.

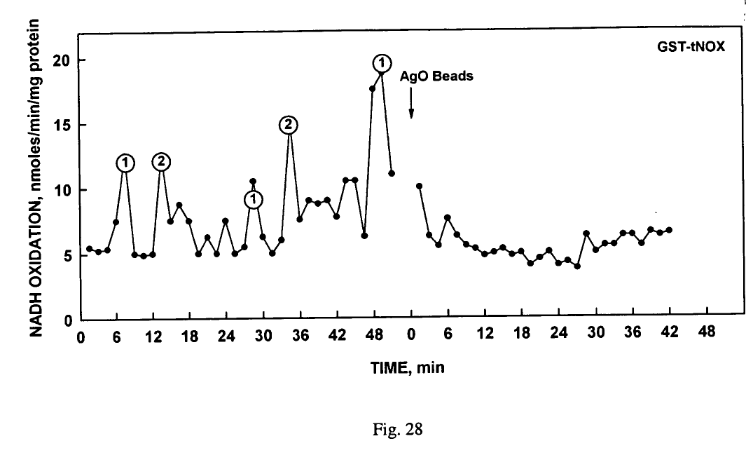

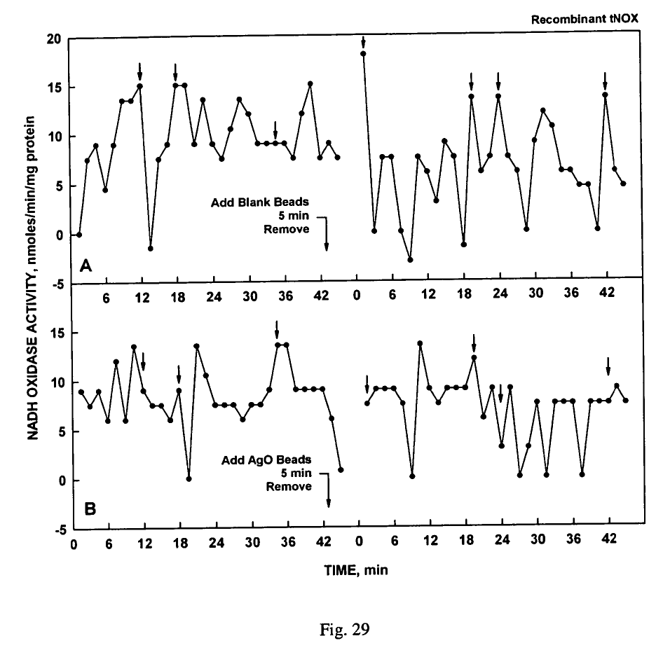

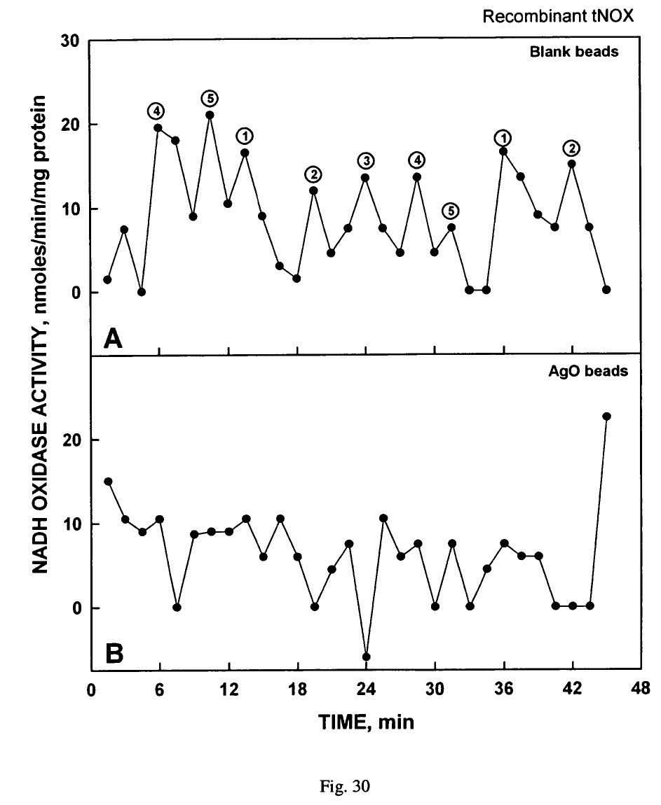

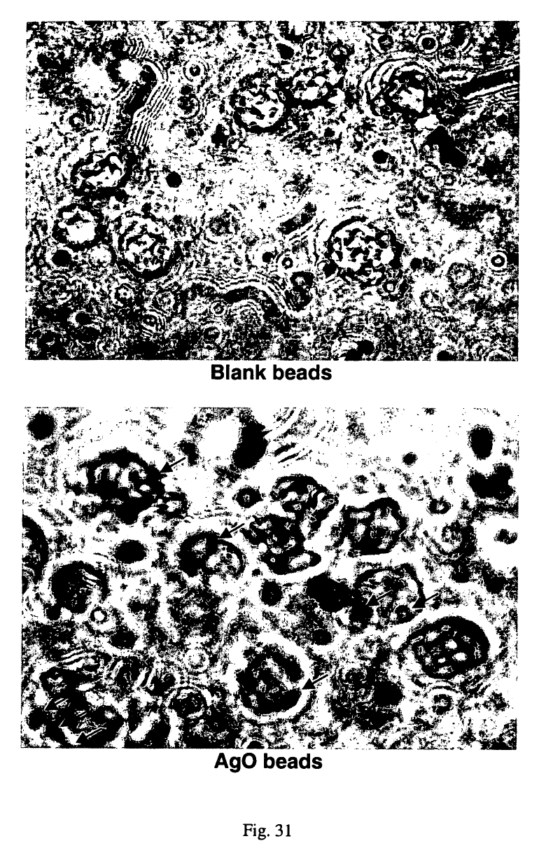

In a further embodiment of the invention, the silver (II) oxide (AgO) may be attached to magnetic beads. An exemplary protocol is provided as Example 2. The beads when added to recombinant tNOX in solution (FIGS. 28 and 29), bound the tNOX and removed it from solution. Blank beads lacking the AgO were without effect on binding tNOX and did not remove the tNOX from solutions (FIGS. 29 and 30). This observation has utility for isolation of tNOX for characterization studies and for binding the magnetic beads to cancer cells that express tNOX on their external surfaces as illustrated for human cervical carcinoma cells (HeLa S) cells in FIG. 31. The beads with AgO were bound to the HeLa cells whereas the blank beads without AgO were not bound. Binding beads to cancer cells through specific combination with tNOX offers opportunities to concentrate cancer cells from mixtures with non-cancer cells such as cancer cells in the circulation (i.e., blood).

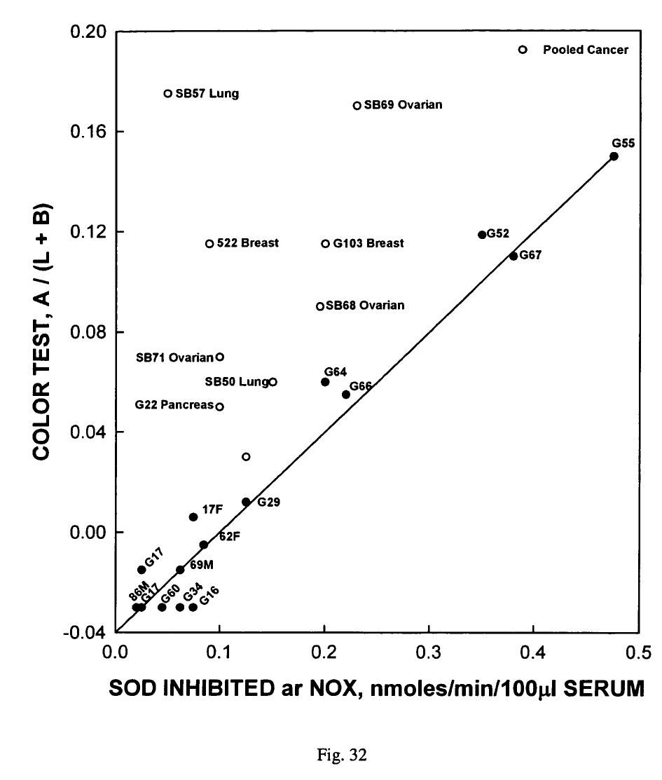

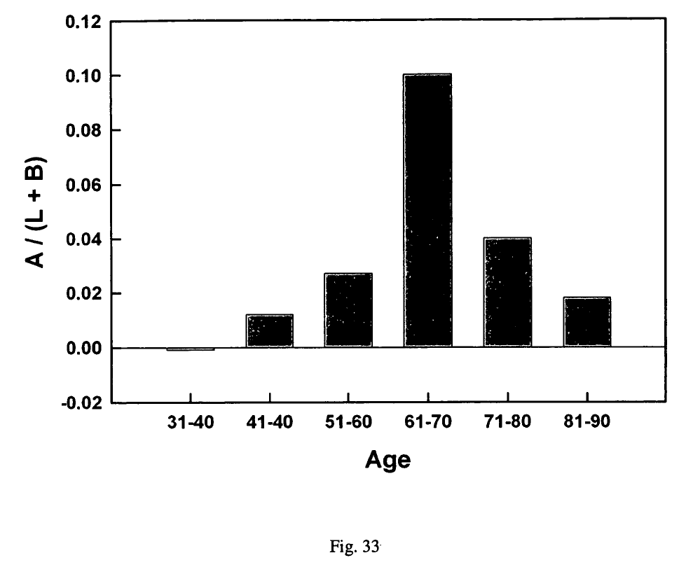

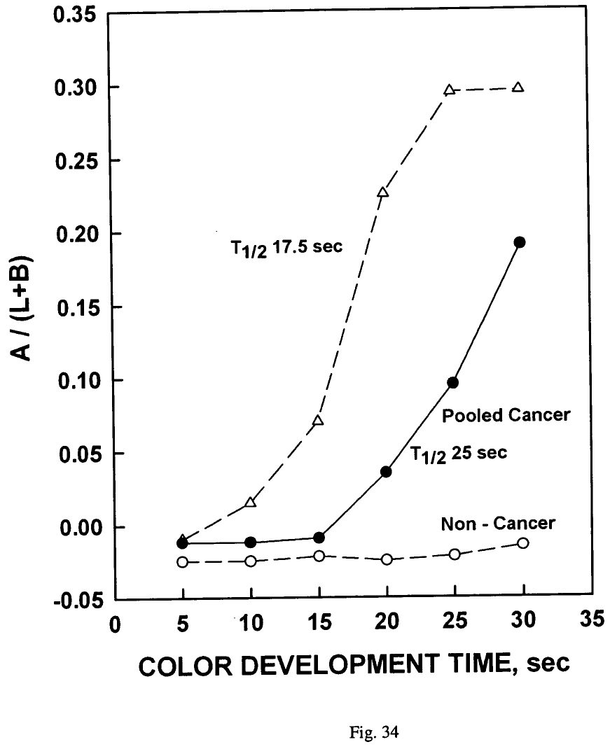

In yet another embodiment, the age-related ECTO-NOX protein (arNOX) also reacts with silver (II) oxide (AgO). With serum samples from individuals not diagnosed as having cancer, especially from older individuals, there is a strong correlation between the A/(L+B) values of the color test and arNOX activity measured by direct assay for superoxide (the product of the enzyme) using reduction of ferricytochrome c as the endpoint (FIG. 32). Thus, arNOX presence may lead to false positives depending on how the serum samples are collected. To reduce the contributions from arNOX, the serum may be centrifuged soon after collection. Under conditions where arNOX is present in the serum, however, the color test may be used in aged individuals as a rapid screen to estimate arNOX levels. Elevated arNOX is especially evident in individuals in the age group 61 to 70 year old most at risk for atherogenic disorders (FIG. 33). Not only does arNOX react with AgO but the reaction appears to occur more readily and more rapidly than with tNOX (FIG. 34).

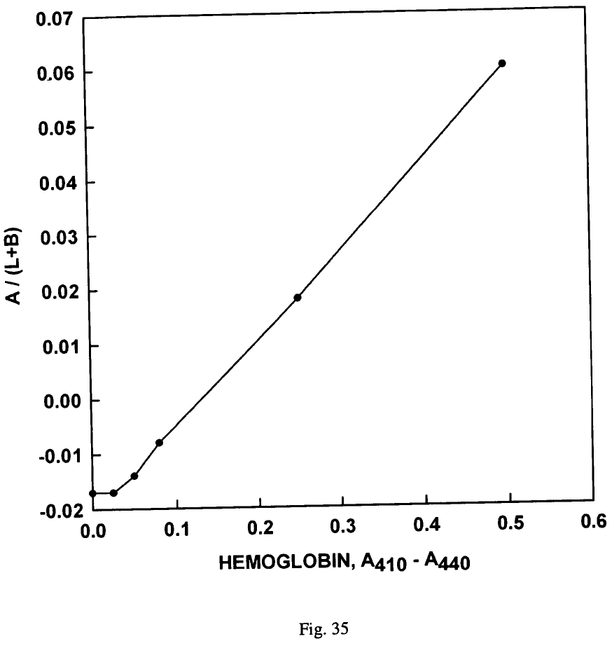

A second potential source of false positives in the color test for cancer is hemolysis resulting from the lysis of red blood cells during collection of serum. We have measured the hemoglobin content of serum from the absorbance at A₄₁₀-A₄₄₀ and when the value exceeds about 0.15, the color test values for non-cancer individuals becomes positive and false positives may result (FIG. 35). This potential source of false positives in the test may be avoided by preventing or reducing hemolysis through extra care in collecting serum samples. However, if hemolysis does occur, the contribution to the color test values may be estimated by determining the absorbance difference at A₄₁₀-A₄₄₀ of the serum from the relationship given in FIG. 35 (Example 14) that correlates this absorbance difference with A/(L+B) values in the color test.

The findings reported below confirm that the color test is not only rapid but both sensitive and reliable. The test thus far is pan cancer (useful for all forms of human cancer) as is the tNOX marker that it is designed to detect. Additionally, the test may be amenable to automation and/or development of self-testing devices.

This invention and embodiments illustrating the method and materials used may be further understood by reference to the following non-limiting examples.

Materials and Methods. Silver (II) Oxide, Item # 22695 (CAS No 1301-96-8) was obtained from Alfa Aesar (Ward Hill, Mass.). All other chemicals were from Sigma or from suppliers indicated.

Growth of cells. Cells were grown in culture in a humidified atmosphere of 5% CO₂ in air at 37° C. Media were renewed every 2 to 3 days. HeLa (ATCC CCL2) cells were grown in 175-cm² flasks in Minimal Essential Medium (Gibco), pH 7.4 at 37° with 10% fetal bovine serum (heat-inactivated), plus 50 mg/L gentamycin sulfate (Sigma). Cells were harvested by scraping and taken up in 140 mM NaCl, 5 mM KCl, 0.7 mM Na₂HPO₄ and 25 mM Tris, pH 7.4, to a final cell concentration of 0.1 g wet weight (gww) per mL. MCF-10A human mammary epithelial cells were cultured in a 1:1 mixture of Ham's F12 medium and Dulbecco's Modified Eagle's medium containing glutamine (Z92 mg/L), hydrocortisone (0.5 μg/mL), EGF (20 ng/mL), and 5% horse serum. Medium was renewed every 2-3 days. BT-20 human breast adenocarcinoma cells were cultured in Eagles minimal essential medium containing 0.1 mM nonessential amino acids with 10% fetal bovine serum and gentamycin sulfate (50 mg/L). Medium was renewed as for MCF-10A cells.

Growth was determined microscopically by counting the number of cells over defined areas consisting of a grid of 1-mm squares. Cell lines were obtained from the American Type Culture Collection.

Spectrophotometric assay of NADH Oxidase. NAD(P)H oxidase activities were measured using paired Hitachi U3210 spectrophotometers over successive 5-minutes intervals (two to four or more at low inhibitory concentrations) at 37° C. The activity was determined as the disappearance of NAD(P)H measured at 340 nm. The reaction mixture included sample, 50 mM Tris-MES, pH 7.0, 2 mM KCN, and 150 μM NAD(P)H in a total volume of 2.5 ml at 37° C. with stirring. A millimolar extinction coefficient of 6.22 was used to determine NAD(P)H disappearance.

Proteins were estimated by the bichinchoninic acid method. Bovine serum albumin was the standard.

Spectrophotometric assay of ECTO-arNOX. ECTO-arNOX was assayed spectrophotometrically as described from the superoxide dismutase inhibited reduction of ferrocytochrome c. Protein was determined by the biochinchoninic acid method with bovine serum albumin as the standard.

Growth measurements. HeLa cell growth was determined using a 96-well plate assay in which cells were fixed with glutaraldehyde and stained with 1% aqueous crystal violet. The absorbance was determined at 580 nm using an automated plate reader.

Analytical SDS-PAGE electrophoresis. Sodium dodecyl sulfate-polyacrylamide gel electrophoresis (SDS-PAGE) was with the buffer system of Laemmli on acrylamide slab gels. The gel was stained for protein using 0.1% Comassie brilliant blue R-250 or silver.

Purification of Plasma Membranes from Cultured Cells. Cultured cells were collected by centrifugation for 6 minutes at 1000 g. The cell pellets were resuspended in 0.2 mM EDTA in 1 mM NaHCO₃ in an approximate ratio of 1 mL per 10⁸ cells and incubated on ice for 10-30 minutes to swell the cells. Homogenization was achieved in 7- to 8-mL aliquots with a Polytron homogenizer (Brinkmann) for 30-40 sec at 10,500 rpm, using a PT-PA 3012/23 or ST-10 probe. To estimate breakage, the cells were monitored by light microscopy before and after homogenization. At least 90% cell breakage without breakage of nuclei was achieved routinely.

The homogenates were centrifuged for 10 minutes at 175 g to remove unbroken cells and nuclei, and the supernatant was centrifuged a second time at 1.4×10⁶ g minutes (e.g., 1 hr at 23,500 g) to prepare a plasma membrane-enriched microsome fraction. The supernatant was discarded, and the pellets were resuspended in 0.2 M potassium phosphate buffer in a ratio of ˜1 mL per pellet from 5×10⁸ cells. The resuspended membranes then were loaded onto the two phase system constituted on a weight basis, consisting of 6.6% (w/w) Dextran T-500 (Pharmacia) and 6.6% (w/w) Polyethylene Glycol 3350 (Fisher) in a 0.2 mM potassium phosphate buffer (pH 7.2) for aqueous two-phase separation. The upper phase, enriched in plasma membranes, was diluted 5-fold with 1 mM sodium bicarbonate and the membranes were collected by centrifugation. The purity of the plasma membrane was determined to be >90% by electron microscope morphometry. The yield was 20 mg of plasma membrane protein from 10¹⁰ cells.

Preparation of HeLa Cells and Cell-Free Extracts. HeLa S cells were collected by centrifugation and shipped frozen in 0.1 M sodium acetate, pH 5, in a ratio of 1 mL of packed cell volume to 1 mL of acetate (Cellex Biosciences). The cells were thawed at room temperature, resuspended, and incutated at 37° for 1 hr to release the protein. The cells were removed by centrifugation at 37,000 g for 60 min, and the cell-free supernatants were refrozen and stored in 1-mL aliquots at −70°.

For heat treatment, 1-mL aliquots of the supernatant material described above were thawed at room temperature and heated to 50° for 10 minutes. The denatured proteins were removed by centrifugation (1500 g, 5 min). Full activity was retained from this step.

For protease treatment, the pH of the heat-stable supernatant was adjusted to 7.8 b addition of 0.1 M sodium hydroxide. Tritirachium album proteinase K (Calbiochem) was added (4 ng/mL) and incubated at 37° for 1 h with full retention of enzymatic activity and drug response. The reaction was stopped either by freezing for determination of enzymatic activity or by addition of 0.1 M phenylmethyl-sulfonyl fluoride in ethanol to yield a final concentration of 10 mM phenylmethylsulfonyl fluoride.

Preparation of Solutions. All solutions are stored at room temperature unless otherwise noted. DDI water is distilled deionized water.

10 mM EGCg (fresh every week). To a 1.5 ml Eppendorf tube add 1000 μl of ethanol. Add 4.6 mg EGCg. Vortex until dissolved. Store at 4° C. and protect from light.

EGCg dilution (fresh every run). To a 1.5 ml Eppendorf tube add 980 μl of ethanol. Add 20 μl of 10 mM EGCg, vortex. To a 0.5 ml add 30 μl of ethanol. Add 20 μl of first EGCg dilution, vortex.

AgO (5 mg/ml soluble, made fresh every run). To a 1.5 ml Eppendorf tube add 980 μl of DDI water. Add in 5 mg of AgO. Add in 20 μl of the final dilution of EGCg. Mix using the pipette tip. Vortex. Centrifuge at 1,000 rpm for 5 sec.

250 mM NiCl₂. To a 15 ml centrifuge tube add 2 ml of DDI water. Add 0.118 g of NiCl₂. Vortex until dissolved.

Silver Enhancer. To a 0.5 ml Eppendorf tube add 1:1 ratio of Initiator to Enhancer (usually 120 μl of each). Vortex for 10 sec.

Initiator (as part of silver enhancer). To a beaker with a stir bar on stir plate add: 80 ml DDI water and 2 g of sodium metabisulfate. Then add in 0.55 g of hydroquinone and 2.07 g of 4-methylaminophenol sulfate. Adjust to a pH of 6.0 with monoethanolamine (usually 500 μl). Add in DDI water to make 100 ml total volume of solution. Pour into a 50 ml tube and wrap with aluminum foil.

Enhancer (as part of silver enhancer). To a beaker with a stir bar on a stir plate add: 80 ml DDI water and 10 g of sodium sulfite. (Solution A) Add a solution of 5 ml water and 1 g sodium metabisulfate to solution A to adjust to pH 7.8 (usually 1 ml). Slowly add in 5 ml DDI water and 0.5 g silver nitrate into solution A. Adjust pH to 7.8 using 5 ml DDI water and 1 gm sodium metabisulfate. Add in DDI water to make 100 ml total volume of solution. Pour into a 50 ml tube and wrap with aluminum foil.

Na₂S₂O3.5H₂O (sodium thiosulfate pentahydrate). To a 125 ml Erlenmeyer flask add 100 ml of DDI water and 2 g of Na₂S₂O₃.5H₂O Swirl until dissolved.

Collection of Blood. For serum analysis, blood is collected by venipuncture in standard BD 5 ml vacutainer SST clot tubes (reference 367986, Cardinal Health) with hemoguard closure. After approximately 30 minutes to allow for clotting, the clot is pelleted by centrifugation for 20 minutes at 3,400 rpm. The clot-free serum is decanted into a clean tube, labeled (date of collection and patient's initials) and analyzed fresh or stored frozen. Alternatively, blood may be collected by venipuncture in standard BD 7 ml vacutainer Thrombin clot tubes with (Catalogue No. 367755) hemoguard closure to reduce hemolysis.

Blood Filtration protocol. A 3 piece filter funnel is placed onto a collection reservoir. Inside the funnel is placed a grade GF/A 1.6 mM glass microfibre filter. Blood is added onto microfibre filter. A vacuum is created inside the collection reservoir to draw blood through the filter into the reservoir. Filters are replaced periodically to avoid clogging pores.

Determination of hemoglobin interference resulting from red cell hemolysis. To correct from hemoglobin coming from hemolysis of red cells to the serum A/(L+B) values, the hemoglobin contribution is determined from the absorbance measured at 420 nm minus the absorbance measured at 440 nm (A₄₂₀-A₄₄₀) and related to A/(L+B) using the relationship of FIG. 35.

### EXAMPLE 1

The following example illustrates a method of identifying the presence of cancer by identifying the presence of tNOX proteins using a silver (II) oxide-based serum color test. To a 0.5 ml Eppendorf tube, the following was added in the order listed: 30 μl soluble AgO with (−)-epigallocatechin-3-gallate (EGCg); 30 μl serum and 30 μl 250 mM NiCl₂. The resulting mixture was incubated at room temperature for 15 minutes in an Eppendorf tube holder. 30 μl of silver enhancer was added and color was allowed to develop for 25 seconds. 30 μl of Na₂S₂O₃.5H₂O (sodium thiosulfate pentahydrate) was then added to stop any reaction. A photograph was taken and color measured.

For 50 non-cancer patients (random out-patient sera from the local hospital for the most part), both female (average age 61, range 18 to 94 years) and male (average age 62, range 9 to 86 years), 4 patients presented with A/(L+B) values that indicated caner presence (a false positive incidence of 8%). Of these, only 1, a 24 year old female was positive in the color test and negative using the electrophoretic (NiAg) test. Of the 50 non-cancer sera, an additional 6 yielded positive A/(L+B) values of 0.025±0.028 (range 0 to 0.05) indicative of incipient cancer.

For 50 cancer patients (uterine, rectal, skin, colon, brain, prostate, ovarian, breast, leukemia and liver), the average A/(L+B) values were 0.171±0.09 and ranged from 0.08 to 0.261. Three serum samples were negative (values of −0.025 to −0.036) and in the normal range. Of these, two appeared normal in an independent test for tNOX, but one, coded as C1, was positive. This gives a confirmed false positive result of 4%. At present, we have no information to understand why C1 was negative in the color test and positive in an alternative test. Certain drugs might interfere with the color test, for example, oral contraceptives, some antibiotics, sulfonamides and MAO inhibitors.

TABLE 1
Dependence of color development with
pooled cancer on ingredients.

| Omissions | A/(L + B) |
|---|---|
| None (Complete) | 0.12-0.15 |
| AgO | No color |
| EGCg | 0.08-0.1 |
| Serum | No color |
| NiCl₂ | 0.08-0.1 |
| Silver enhancer | No color |
| Sodium thiosulfate | Reaction continues* |

*Differences among samples eventually obscured.

### EXAMPLE 2

This example illustrates a method of Derivatizing Polystyrene-Coated Magnetic Beads with Silver (II) Oxide in accordance with the present invention. Magnetic beads were from Bangs Laboratories, Estapor SuperParaMagnetic Microspheres Catalog Code: ME03N. Description: P(S/V-COOH)Mag/Encapsulated((42.5%); Brown; Mean Diameter: 1.63 μm; Quantity: 0.5 g; Store at: 2-8° C.; Solids: 10%; Inv#: L050309A; Bangs Lot# 3734 (Made in EC).

Washing of Magnetic Beads before Derivatization. 50 μl of stock bead solution (from Bangs Laboratories) was placed in 1.5 ml micro-centrifuge tube. 450 μl of Tri-Mes buffer, pH 9 (made with DDI water) was added. Beads were collected by centrifugation for 2 minutes at 5,000 rpm. Buffer solution was removed and washes were repeated three times. Beads were resuspended by pipetting up and down (vortexing traps beads in foam). The last wash was resuspended in 450 μl DDI water.

Derivatization of Magnetic Beads with AgO. 50 μl of washed bead solution was placed in 1.5 ml micro-centrifuge tube in 450 μl DDI H₂O. 430 μl of a 0.1% to 1% acetone solution made in DDI water was added. 20 μl of a saturated silver oxide solution made in DDI water (˜5 mg AgO in 1 ml DDI water) was added and mixed by pipetting. Solution was allowed to stand for ˜1-2 minutes. Beads were washed 4 times with DDI water (sample washing procedure as above) and resuspended in DDI water as required.

Although the present invention has been described in considerable detail with reference to certain preferred embodiments thereof, other versions are possible. Therefore the spirit and scope of the appended claims should not be limited to the description and the preferred versions contained within this specification.

What is claimed is:

1. A method of detecting the presence of cancer comprising:
providing a sample from a patient;
providing silver (II) oxide to the sample;
determining the presence of color in the sample.

2. The method of claim 1 further comprising providing (−)-epigallocatechin-3-gallate.

3. The method of claim 1 further comprising providing nickel chloride.

4. The method of claim 1 further comprising incubating the sample.

5. The method of claim 4, wherein the sample is incubated for about 15 minutes.

6. The method of claim 1 further comprising providing a silver enhancer.

7. The method of claim 6 further comprising providing a stop reagent.

8. The method of claim 7, wherein said stop reagent is sodium thiosulfate pentahydrate.

9. The method of claim 1, wherein the sample is selected from serum, blood, plasma, tissue, saliva and urine.

10. The method of claim 1, wherein the sample is centrifuged.

11. The method of claim 10, wherein the sample is centrifuged prior to provide the silver (II) oxide.

12. A method of detecting the presence of cancer comprising:
obtaining serum from a patient;
contacting the serum with silver (II) oxide to form a mixture; and
determining the presence of color in the mixture.

13. The method of claim 12 further comprising centrifuging the serum prior to contacting the serum with silver (II) oxide.

14. The method of claim 12 further comprising mixing (−)-epigallocatechin-3-gallate with the silver (II) oxide.

15. The method of claim 12 further comprising mixing nickel chloride with the serum.

16. The method of claim 12 further comprising incubating the mixture.

17. The method of claim 16 further comprising mixing silver enhancer with the mixture.

18. The method of claim 17 further comprising mixing a stop reagent with the mixture.

19. The method of claim 18, wherein said stop reagent is sodium thiosulfate pentahydrate.

20. An assay for detecting or monitoring abnormal cellular proliferation comprising:
silver (II) oxide.

21. The assay of claim 20 further comprising (−)-epigallocatechin-3-gallate.

22. The assay of claim 20 further comprising nickel chloride.

23. The assay of claim 20 further comprising a silver enhancer.

24. The assay of claim 20 further comprising a stop reagent.

25. The assay of claim 24, wherein the stop reagent is sodium thiosulfate.

26. A method of detecting the presence of cancer comprising:
obtaining a sample from a patient;
contacting the sample with silver (II) oxide; and
detecting binding of the silver (II) oxide to a cancer-associated protein.

27. The method of claim 26, wherein the cancer-associated protein is selected from tNOX, arNOX and combinations thereof.

28. The method of claim 26, wherein the sample is selected from tissue, plasma, serum, blood, urine, and saliva.

29. A method of measuring the progression of cancer comprising:
obtaining a sample;
contacting the sample with silver (III) oxide; and
detecting the binding of silver (II) oxide to arNOX.

30. The method of claim 29 further comprising adding (−)-epigallocatechin-3-gallate.

31. The method of claim 29 further comprising adding nickel chloride.

32. The method of claim 29 further comprising adding a silver enhancer.

33. The method of claim 29 further comprising adding a stop reagent.

34. The method of claim 33, wherein the stop reagent is sodium thiosulfate.

35. A method of assaying cancer cells in vivo comprising:
administering silver (II) oxide bound to magnetic beads to a subject; and
detecting the magnetic beads.
